(19) United States
(12) Patent Application Publication
(10) Pub. No.: US 2024/0081360 A1
(43) Pub. Date: Mar. 14, 2024

(54) BASE OIL FOR OILS AND FATS USED IN FUNCTIONAL FOOD, PREPARATION METHOD AND USE THEREOF

(71) Applicant: Nanchang University, Nanchang (CN)

(72) Inventors: Zheling Zeng, Nanchang (CN); Guibing Zeng, Nanchang (CN); Zhen Ouyang, Nanchang (CN); Bo Yang, Nanchang (CN); Ping Yu, Nanchang (CN); Jiaheng Xia, Nanchang (CN); Maomao Ma, Nanchang (CN); Dongman Wan, Nanchang (CN); Miao Luo, Nanchang (CN); Cheng Zeng, Nanchang (CN); Xuefang Wen, Nanchang (CN)

(73) Assignee: Nanchang University, Nanchang (CN)

(21) Appl. No.: 18/273,751

(22) PCT Filed: Dec. 31, 2021
(86) PCT No.: PCT/CN2021/143605

§ 371 Date: Nov. 10, 2023

(30) Foreign Application Priority Data
Jan. 22, 2021 (CN) 202110086345.3

(51) Int. Cl. A23D 7/01; A23D 7/04; A23D 9/013; C12N 9/20; C12P 7/6454

(52) U.S. Cl. CPC A23D 7/01; A23D 7/04; A23D 9/013; C12N 9/20; C12P 7/6454; C12Y 301/01003

(57) ABSTRACT

Disclosed is a base oil for functional food oils and fats, a preparation method therefor and the use thereof. The base oil for functional food oils and fats is formed through ternary transesterification on medium-chain triglycerides, high-melting-point fat and oils rich in linolenic acid. The base oil for functional food oils and fats has a wide melting range, can significantly improve the glucose and lipid metabolism disorder, balance the essential and functional fatty acids in the body, and quickly replenish energy. Animal experiments were conducted and the fatty acid composition and distribution of the base oil were optimized and determined through comparative analysis based on evaluation indicators such as the improved effect in glucose and lipid metabolism and melting point.

### CROSS-REFERENCE TO RELATED APPLICATION

This patent application is a national stage application of International Patent Application No: PCT/CN2021/143605, filed on Dec. 31, 2021, which claims the benefit and priority of Chinese Patent Application No. 202110086345.3 filed with the China National Intellectual Property Administration on Jan. 22, 2021, the disclosure of which is incorporated by reference herein in its entirety as part of the present application.

### TECHNICAL FIELD

The present disclosure belongs to the technical field of edible oils and fats and relates to base oil for oils and fats used in functional food and a preparation method thereof.

### BACKGROUND

Based on the length of carbon chain, fatty acids can be divided into short-chain fatty acids (fatty acid having 2-6 carbon atoms, abbreviated as SCFA), medium-chain fatty acids (fatty acids having 8-12 carbon atoms, abbreviated as MCFA), and long-chain fatty acids (fatty acids having more than 12 carbon atoms, abbreviated as LCFA). Depending on whether fatty acids can be synthesized in human body, fatty acids can be categorized into essential fatty acids (abbreviated as EFA) and non-essential fatty acids (abbreviated as NEFA).

Oils and fats are mixed fatty acid glycerides, which can be divided into long-chain triglycerides (abbreviated as LCT), medium and long-chain triglycerides (abbreviated as MLCT), medium-chain triglycerides (abbreviated as MCT), and short-chain triglycerides (abbreviated as SCT) based on the carbon numbers of the fatty acids attached to the backbone of the fatty acid glycerides.

Oils and fats are one of the three classes of major energy-producing nutrients and one of classes of the six major nutrients for human body. The energy per unit of oils and fats (9 kcal) is 2.25 times that of the other two class of energy-producing nutrients carbohydrate (4 kcal) and protein (4 kcal). Because most oils and fats contain essential fatty acids needed by the human body, if the human body lacks fat over a long time, it will lead to serious physiological disorders.

Research reports on long-chain triglycerides and long-chain fatty acids at home and abroad show that long-chain triglycerides are absorbed, transported and stored in the body in the form of triglyceride. The transport and metabolic energy of the long-chain fatty acids in cells depends on the carnitine-acylcarnitine transferase system, and the long-chain fatty acids have a large molecular weight, low solubility in blood, long half-life, slow and incomplete metabolism and clearance. Excess long-chain fatty acids in the body are readily re-esterified into long-chain glycerides and accumulated in blood, liver, adipose tissue and other tissues, affecting the functions of liver, kidney, lung and other organs, causing glucose and lipid metabolism disorder. Long-term and excessive intake of high-energy food rich in long-chain triglycerides is one of the main causes of metabolic syndromes such as overweight, obesity, non-alcoholic fatty liver disease (NAFLD), hyperlipidemia, hyperglycemia, hypertension, hyperviscosity, hyperuricemia, and hyperinsulinemia.

Research reports on medium-chain triglycerides and medium-chain fatty acids at home and abroad show that the medium-chain fatty acids contained in medium-chain triglycerides include caprylic acid (abbreviated as C), capric acid (abbreviated as Ca), and lauric acid (abbreviated as La). Medium-chain triglycerides are absorbed, transported and metabolized in the body in a form of free medium-chain fatty acids. Medium-chain fatty acids have small molecular weight, high solubility in blood, and short half-life. The transport of medium-chain fatty acids in the body does not need to rely on the carnitine-acylcarnitine transferase system and the medium-chain fatty acids can directly enter into the cells and mitochondria for energy production via oxidation. Medium-chain fatty acids undergo rapid metabolism and energy production in the body, leading to rapid and complete clearance in blood. Medium-chain fatty acids are not prone to be re-esterified in the body, have little effect on liver, kidney, lung and other organs, do not compete with bilirubin for albumin, do not exacerbate jaundice, and have a more significant effect of saving protein (nitrogen) than the long-chain fatty acids. Medium-chain triglycerides can quickly replenish energy in the body and mitigate glucose and lipid metabolism disorder in the body. However, medium-chain fatty acids are non-essential fatty acids for the human body, nor can they be converted into functional fatty acids in the body or supply essential fatty acids and functional fatty acids needed for human growth and development.

The linoleic acid (abbreviated as L) in long-chain fatty acids is a kind of omega-6 essential fatty acids, and the linolenic acid (abbreviated as Ln) is a kind of omega-3 essential fatty acids. Linoleic acid and linolenic acid are precursors or parents for synthesis of unsaturated fatty acid such as arachidonic acid (abbreviated as ARA), eicosapentaenoic acid (abbreviated as EPA), docosapentaenoic acid (DPA), Docosahexaenoic acid (abbreviated as DHA), prostaglandin (abbreviated as PG), thromboxane (abbreviated as TXA) and leukotrienes (abbreviated as LT) in the body. These polyunsaturated fatty acids are important components of the brain and retina, which can promote and maintain the development and growth of the brain nervous system and visual system, reduce blood triglyceride and cholesterol levels, prevent cholesterol and fat from accumulating on the artery wall, make cardiovascular and cerebrovascular system and immune system healthy, and alleviate glucose and lipid metabolism disorder in the body.

The vast majority of natural edible fats are long-chain triglycerides having a content of long-chain fatty acids up to 95% (w/w), such as soybean oil, palm oil, peanut oil, rapeseed oil, lard oil, corn oil, rice bran oil, tea seed oil, olive oil, cocoa butter, etc. The only medium and long-chain triglycerides having a content of more than 50% of medium-chain fatty acids are coconut oil (about 7.5% caprylic acid, 7.0% capric acid, 48.0% lauric acid), palm kernel oil (about 3.9% caprylic acid, 5.0% capric acid, 47.5% lauric acid), Litsea cubeba kernel oil (about 15.8% capric acid, 71.6% lauric acid), the medium-chain triglycerides having a content of more than 95% (w/w) of medium-chain fatty acids are only the Cinnamomum camphora seed kernel oil (abbreviated as CCSKO) (containing caprylic acid 0.32-0.47%, capric acid 56.49-61.98%, lauric acid 34.18-39.20%) discovered by the inventor of the present disclosure, and short-chain triglycerides having a content of greater than 1% of fatty acids is the butterfat produced from fermented milk.

Food-dedicated oils and fats used in cold drinks (ice cream, popsicle) and beverages (milk tea, meal replacement powder, coffee), for example, margarine and powdered oil, are still produced using long-chain triglycerides such as animal fat, hydrogenated vegetable oil, and palm oil and medium and long-chain triglycerides with capric acid and caprylic acid content less than 30% w/w such as palm kernel oil, and coconut oil as raw material. As a result, food-specific oils still have the deficiencies of being rich in long-chain triglycerides, containing trans-fatty acids, lower content of medium-chain fatty acids, and lacking essential fatty acids (linolenic acid and linoleic acid). Consumers consuming foods containing these food-dedicated oils and fats are vulnerable to metabolic syndrome diseases such as overweight, obesity, non-alcoholic fatty liver disease, hyperlipidemia, hyperglycemia, hypertension, hyperviscosity, hyperuricemia, and hyperinsulinemia.

In order to overcome the disadvantages of food-dedicated oil products rich in long carbon chain fatty acids, relevant oil researchers worldwide have successively developed a variety of medium and long carbon chain food-dedicated oils and fats and their preparation methods, mainly including the methods as follows.

Patent application (CN201310261549) discloses an oil composition used for margarine and shortening containing medium-chain fatty acid triglyceride, long-chain fatty acid triglyceride or medium long-chain fatty acid triglyceride and a preparation method thereof. Based on the total weight of the composition, the oil composition contains more than 80 wt % triglyceride, and based on the total weight of all fatty acids forming the oil composition, medium-chain fatty acids account for 8 to 15 wt %. The oil composition is obtained by physically mixing or transesterification, wherein the transesterification can be chemical transesterification or enzymatic transesterification.

Patent application CN201310410498 discloses an oil composition for non-dairy creamer and non-dairy creamer prepared therefrom, in particular relates to an oil composition for non-dairy creamer with zero trans-fatty acid and non-dairy creamer prepared therefrom. The oil composition for the zero trans-fatty acid non-dairy creamer is characterized in that in all fatty acids constituting the oil composition, the medium-carbon chain fatty acids and long-chain fatty acids have a total weight percent of more than 98 wt %, and the weight ratio of medium-chain fatty acids and long-chain fatty acids is 10-35:65-90. The non-dairy creamer prepared by using the oil composition for the zero trans-fatty acid non-dairy creamer of the present disclosure solves the problem of the harm to the body resulting from excessive trans-fatty acid content in the traditional non-dairy creamer made from hydrogenated vegetable oil.

Patent application CN201310717548 discloses a dedicated oil for ice cream, which is characterized in that, the medium-chain fatty acids account for 5%-25% relative to the total weight of all fatty acids forming the dedicated oil and the dedicated oil contains 75%-95% component B; wherein, the medium-chain fatty acids are fatty acids with 6 to 10 carbon, and the component B is selected from palm oil, palm kernel oil, and modified products thereof. The present disclosure also provides an ice cream composition prepared from the ice cream dedicated fat of the present disclosure.

US Patent application US2004/0191391A1 discloses cooking oil with a medium-chain fatty acid content of 5-23% and a mass content of 1-20% of triglyceride containing two medium-chain fatty acids molecules.

Patent application CN106490189A discloses margarine prepared from functional oils with low oil content and no trans-fatty acids, in which the content of medium-chain triglycerides and other functional oils is 5-9%.

Patent application CN103315071A discloses a method of preparing vegetable oil whipped cream using formulated non-hydrogenated vegetable oil. The content of palm kernel oil and coconut oil used in this patent application is 18% to 30%, and the amount of essential fatty acids is not considered.

Patent application JP2015211666A discloses that a medium-chain fatty acid triglyceride and long-chain fatty acid triglyceride are used to prepare margarine after transesterification in a certain ratio, wherein the content of medium-chain triglycerides used is from 0.5% to 100%, with the dosage of essential fatty acids not being considered.

All of the products disclosed in the above patent applications have the problems of the content of medium-chain fatty acids in medium and long carbon chain food dedicated fats being lower than 30% (w/w), the contained medium-chain fatty acids being all caprylic acid, capric acid, and essential fatty acids (linolenic acid and linoleic acid) being low and unreasonable composition ratio.

Therefore, it is extremely important to prepare a base oil (abbreviated as BO) for dedicated trigylceride for functional foods, in which medium-chain fatty acid accounting for fatty acid total mass ratio is more than 65%, and linoleic acid than linolenic acid mass ratio is 0.5, and meets the requirement for dedicated trigylceride for functional food.

### SUMMARY

The objects of the present disclosure are achieved by the following technical solutions.

The first object of the present disclosure is to provide a base oil for functional food oils and fats, wherein the base oil for functional food oils and fats formed through transesterification by using Cinnamomum camphora seed kernel oil or mixed oils and fats having similar components to fatty acids in Cinnamomum camphora seed kernel oil as a main raw material and using high-melting-point fats such as basa catfish solid fraction or palm stearin and oils rich in linolenic acid as auxiliary raw materials. The base oil for functional food oils and fats has the characteristics of wide melting range, can significantly improve glucose and lipid metabolism disorder in the body, balance and replenish essential fatty acids and functional fatty acids in the body, and quickly replenish energy in the body.

The base oil for functional food oils and fats described in the present disclosure is formed by transesterification of medium-chain triglycerides, high-melting-point fats, and oils rich in linolenic acid.

The medium-chain triglycerides include Cinnamomum camphora seed kernel oil, and mixed oils and fats having similar components to fatty acids in Cinnamomum camphora seed kernel oil.

The high-melting-point fats have a melting point ranging from 44 to 52° C., including basa catfish solid fraction, palm stearin and the like.

The oils rich in linolenic acid include Perilla seed oil, linseed oil and the like.

Based on the mass of fatty acid in the fatty acid of the base oil for functional food oils and fats described in the present disclosure, the mass percent of medium-chain fatty acids is 63% to 69%, and the mass ratio of linoleic acid and linolenic acid in long-chain fatty acids is 0.5. Preferably, the medium-chain fatty acids in the base oil for functional food oils and fats accounted for 65% by mass of the total fatty acid.

The medium-chain fatty acids are derived from Cinnamomum camphora seed kernel oil or mixed oils and fats having similar components to fatty acids in Cinnamomum camphora seed kernel oil. Long-chain fatty acids are derived from fats having a melting point of 44-52° C. such as basa catfish solid fraction or palm stearin and oil rich in linolenic acid.

Preferably, the medium-chain triglycerides used in the base oil for functional food oils and fats described in the present disclosure come from Cinnamomum camphora seed kernel oil.

When calculating the SFC at 25° C. and 30° C., the base oil for functional food oils and fats described in the present disclosure has an SFC of 5.8%-15.6% at 25° C. and 0%-8.3% at 30° C., respectively. Preferably, the SFC of the base oil for functional food oils and fats described is 13.8% at 25° C. and 7.5% at 30° C., respectively.

The inventors of the present disclosure found that Cinnamomum camphora seed kernel oil (abbreviated as CCSKO) contains about 0.32-0.47% caprylic acid, 56.49-61.98% capric acid, and 34.18-39.20% lauric acid, and the content of the medium-chain fatty acids is equal to or greater than 95%, which belongs to the natural medium-chain triglyceride.

The second object of the present disclosure is to provide a method for preparing the above-mentioned base oil for functional food oils and fats.

The method for preparing the base oil for functional food oils and fats described in the present disclosure, comprises the following steps of: subjecting medium-chain triglycerides and high-melting-point fats, oils rich in linolenic acid to ternary transesterification at a proper temperature and at a proper strength of stirring, using lipase as a catalyst, to obtain the base oil for functional food oils and fats at one step. In the method, the mass percent of the medium-chain fatty acids in the base oil for functional food oils and fats is 63% to 69%, and the mass ratio of linoleic acid to linolenic acid in the long-chain fatty acids is 0.5.

Preferably, the mass percent of the medium-chain fatty acids in the medium-chain triglycerides in the base oil for functional food oils and fats is 65%.

The lipase is selected from the group consisting of Lipozyme RM IM, Lipozyme TL IM, Novozyme 435, and Staphylococcus caprae lipase. Preferably, the lipase is Staphylococcus caprae lipase.

The addition amount of lipase is 5-25% based on the mass of the mixed oil, a temperature for ternary transesterification reaction is 35-55° C., and a time for ternary transesterification reaction is 1-8 h. Preferably, the addition amount of lipase is 10% based on the of mass of the mixed oil, a temperature for the ternary transesterification reaction is 50° C., and a time for ternary transesterification reaction is 4h.

The third object of the present disclosure is the use of the above-mentioned base oil for functional food oils and fats in food.

The above-mentioned food includes but not limits to powdered oil, margarine, sports nutrition food.

The base oil for functional food oils and fats described in the present disclosure has a wide melting range, can significantly improve the glucose and lipid metabolism disorder in the body, balance the essential and functional fatty acids in the body, and quickly replenish energy. The base oil for functional food oils and fats of the present disclosure can satisfy the needs of dietary and nutritional needs of the consumers, especially patients with metabolic syndrome such as obesity, non-alcoholic fatty liver disease, hyperlipidemia, hyperglycemia, hypertension, hyperviscosity, hyperuricemia, hyperinsulinemia and athletes and the base oil for functional food oils and fats can be widely used in margarine, powdered oil, and sports nutrition food and can improve human health and living standards, having significant social, ecological and economic benefits.

### BRIEF DESCRIPTION OF THE DRAWINGS

FIG. 1A-FIG. 1D show the effects of the mass percentage of medium-chain fatty acids accounting for the total fatty acid in the base oil for functional food oils and fats on various indicators of obesity model mice in Example 1; wherein, FIG. 1A is the effect on the weight of mouse, FIG. 1B shows the effect on fat coefficient in mouse, FIG. 1C shows the effect on serum triglyceride (TG) in mouse serum; and FIG. 1D shows the effect on total cholesterol (TC) in mouse serum.

FIG. 2A-FIG. 2D show the effects of the mass percentage of medium-chain fatty acids accounting for the total fatty acid in the base oil for functional food oils and fats on various indicators of obesity model mice in Example 1; wherein, FIG. 2A shows the effect on low-density lipoprotein (LDL-C) in mouse serum, FIG. 2B shows the effect on high-density lipoprotein (HDL-C) in mouse serum, FIG. 2C shows the effect on fasting blood glucose (FBG) in mouse serum, and FIG. 2D shows the effect on mouse serum fasting insulin (FINs) in mouse serum.

FIG. 3A-FIG. 3C show the effects of the mass percentage of medium-chain fatty acids accounting for the total fatty acid in the base oil for functional food oils and fats on various indicators of obesity model mice in Example 1; wherein, FIG. 3A shows the coefficient of insulin resistance (HOMA-IR) in mouse, FIG. 3B shows the effect on alanine aminotransferase (ALT) in mouse serum, and FIG. 3C shows the effect on aspartate aminotransferase (AST) in mouse serum.

FIG. 4 shows the effects of the mass percentage of medium-chain fatty acids accounting for the total fatty acid in the base oil for functional food oils and fats on SFC at different temperatures.

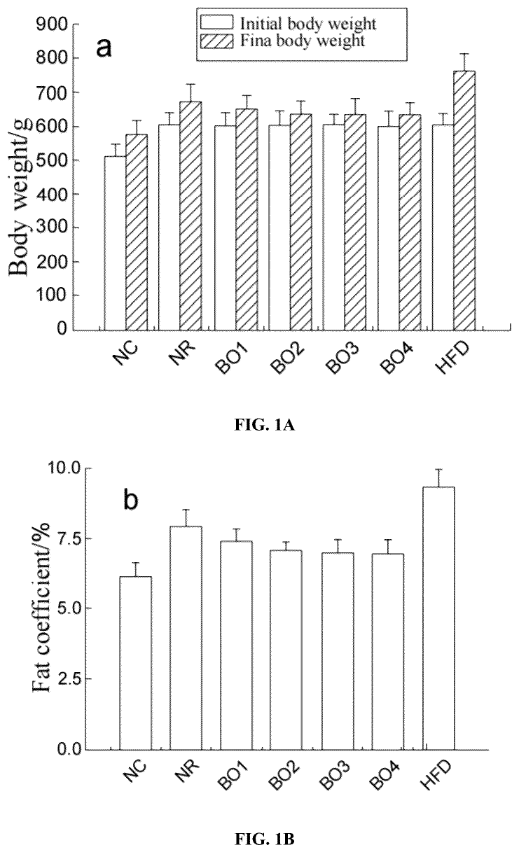

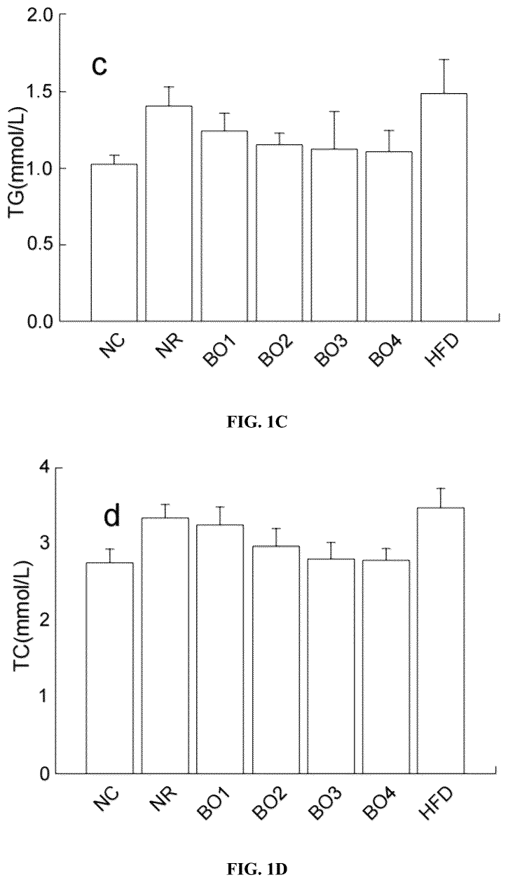

In FIG. 1A-FIG. 1D and FIG. 4, H-BO—feed containing high-fat base oil for functional food oils and fats, NC—normal chow (AIN-93M) group, NR—recovery group, RFD—high-fat feed (D12451) group, BO1—H-BO group in which MCFA accounts for 63% by mass of the total fatty acid, BO2—H-BO group in which MCFA accounts for 65% by mass of the total fatty acid, BO3—H-BO group in which MCFA accounts for 67% by mass of the total fatty acid, and BO4—H-BO group in which MCFA accounts for 69% by mass of the total fatty acid.

### DETAILED DESCRIPTION OF THE EMBODIMENTS

The present disclosure will be further described below in conjunction with specific examples. Unless otherwise indicated specifically, the experimental methods in the following examples are generally carried out using conventional conditions. Unless otherwise indicated, all percentages, proportions, or ratios are calculated in mass.

Unless otherwise specified, all professional and scientific terms used in the examples have the same meanings as commonly understood by those skilled in the art. In addition, any methods and materials similar or equivalent to those described can be used in the present disclosure. The preferred embodiments and materials described in the examples are for exemplification purposes only.

In the following examples of the present disclosure, reference can be made to related standards or journals.

For the method of testing content of fatty acids, reference can be made to China national standard GB 5009.168-2016.

For the method of testing transesterification rate, reference can be made to Characterization of medium-chain triacylglycerol (MCT)-enriched seed oil from Cinnamomum camphora (Lauraceae) and its oxidative stability; (Journal of Agricultural and Food Chemistry, 2011, 59(9):4771-4778).

For the method of testing the content of Sn-2 fatty acids, reference can be made to China national standard GB/T 24894-2010 and GB 5009.168-2016.

For the method of testing the solidification point, reference can be made to Standard SN/T0801.17-2010.

GC model: Agilent7890B Column: DB-23 fused silica glass capillary column (30m*0.25 mm*0.25 μm).

HPLC model: Agilent1260 Chromatographic column: C18 column (5 μm*4.6 mm*200 mm).

In the following examples of the present disclosure, the Cinnamomum camphora seed kernel oil is self-made, and the basa catfish solid fraction, palm stearin, Perilla seed oil, and linseed oil used were all purchased from the market; lipase Lipozyme RM IM was purchased from Novozymes Biotechnology Co., Ltd., lipase Lipozyme TL IM was purchased from Novozymes Biotechnology Co., Ltd., lipase Novozyme 435 was purchased from Novozymes Biotechnology Co., Ltd., and lipase Staphylococcus caprae lipase was self-made.

In Example 1 of the present disclosure, the feeds used in the animal experiments were normal chow (AIN-93M), high-fat feed (D12451) and high-fat feed containing base oil for functional food oils and fats (H-BO), and the formula for these feeds are shown in Table 1-1 and Table 1-2 and calorification rate.

TABLE 1-1
Formula of normal chow and high-fat diet in animal experiments

| Feed type | | | | |
|---|---|---|---|---|
| Energy-producing | Normal chow (AIN-93M) | | High-fat feed (D12451) | |
| component | gm % | Kcal % | gm % | Kcal % |
| Protein | 14.20 | 14.70 | 24.00 | 20.00 |
| Carbohydrate | 73.10 | 75.90 | 41.00 | 35.00 |
| Fat | 4.00 | 9.40 | 24.00 | 45.00 |
| wherein: medium-chain | — | — | — | — |
| fatty acids | | | | |

| Components | gm | Kcal | gm | Kcal |
|---|---|---|---|---|
| Casein | 140.00 | 560.00 | 233.06 | 932.24 |
| L-cystine | 1.80 | 7.20 | 3.50 | 14.00 |
| Corn starch | 495.70 | 1983.00 | 84.83 | 339.32 |
| Maltodextrin 10 | 125.00 | 500.00 | 116.53 | 466.12 |
| Sucrose | 100.00 | 400.00 | 201.36 | 805.44 |
| Cellulose | 50.00 | — | 58.27 | — |
| Soybean oil | 40.00 | 360.00 | 29.13 | 262.17 |
| Lard oil | — | — | 206.84 | 1861.56 |
| Base oil for functional | — | — | — | — |
| food oils and fats | | | | |
| Mineral mix | 35.00 | — | 11.65 | — |
| Calcium hydrogen | — | — | 15.15 | — |
| phosphate | | | | |
| Calcium carbonate | — | — | 6.41 | — |
| Potassium citrate, | — | — | 19.23 | — |
| 1H₂O | | | | |
| Vitamin addition | 10.00 | 40.00 | 11.65 | 46.60 |
| Choline Bitartrate | 2.50 | — | 2.33 | — |
| Pigment Red #40 | — | — | 0.06 | — |
| Total | 1000.00 | 3850.00 | 1000.00 | 4727.45 |

TABLE 1-2
Formula of high-fat feed containing base oil for functional
food oils and fats used in the animal experiments

| | |
|---|---|
| Feed type | High-fat Base oil for functional |
| Energy-producing | food oils and fats (H-BO) |

| component | gm % | Kcal % |
|---|---|---|
| Protein | 24.00 | 20.00 |
| Carbohydrate | 41.00 | 35.00 |
| Fat | 24.00 | 45.00 |

| Components | gm | Kcal |
|---|---|---|
| Casein | 233.06 | 932.24 |
| L-cystine | 3.50 | 14.00 |
| Corn starch | 84.83 | 339.32 |
| Maltodextrin 10 | 116.53 | 466.12 |
| Sucrose | 201.36 | 805.44 |
| Cellulose | 58.26 | — |
| Soybean oil | — | — |
| Lard oil | — | — |
| Base oil for functional | 235.97 | 2123.73 |
| food oils and fats | | |
| Mineral mix | 11.65 | — |
| Calcium hydrogen | 15.15 | — |
| phosphate | | |
| Calcium carbonate | 6.41 | — |
| Potassium citrate, | 19.23 | — |
| 1H₂O | | |
| Vitamin addition | 11.65 | 46.60 |
| Choline Bitartrate | 2.33 | — |
| Pigment Red #40 | 0.06 | — |
| Total | 1000.00 | 4727.45 |

In the Example 1 embodiment of the present disclosure, compared with the obesity model mice in the high-fat feed group, the base oil for functional food oils and fats had the effect of significantly improving the glucose and lipid metabolism disorder in the body, which means that it improved the effect of glucose and lipid metabolism disorder in the obesity model mice by 15% or above. That was, the levels of the indicators such as fat coefficient, serum triglyceride (TG), serum total cholesterol (TC), serum low-density lipoprotein (LDL-C), serum high density lipoprotein (HDL-C), fasting blood glucose (FGB), fasting insulin (FINs), insulin resistance coefficient (HOMA-IR=[(FBG(mmol/L)×FINs(ng/ml)]/22.5), aspartate aminotransferase (AST), alanine aminotransferase (ALT) and other index levels decreased or increased by 15% or above.

### Example 1

In this Example, the fatty acids from the Cinnamomum camphora seed kernel oil, the basa catfish solid fraction, and the Perilla seed oil were used as raw materials, and their composition and distribution are shown in Table 2. Based on a mass percentage of medium-chain fatty acids being 63%, 65%, 67%, 69%, and a mass ratio of linoleic acid to linolenic acid being 0.5, an appropriate amount of Cinnamomum camphora seed kernel oil, soybean oil, and linseed oil were weighed and placed in different esterification reactors, and Staphylococcus caprae lipase was added according to a percentage of 10% (w/w) of the mixed oil. The reaction temperature was 50° C., and the reaction time was 4 h under stirring. After the ternary transesterification reaction was completed, the lipase was isolated, and the ternary transesterification rate and SFC (solid fat coefficient) were measured to obtain A series of base oil for functional food oils and fats, with the mass percentage of medium-chain fatty acids accounting for the total fatty acid being 63%, 65%, 67%, and 69%, respectively, the mass ratio of linoleic acid to linolenic acid being 0.5, the ternary transesterification rates being 73.13%, 74.05%, 74.35%, 73.91%, the SFC at 25° C. being 15.6%, 13.8%, 9.7%. 5.8%, and the SFC at 30° C. being 8.3%, 7.5%, 3.8%, and 0%, respectively. The composition of fatty acids in base oil for functional food oils and fats with different mass percentages of medium-chain fatty acids accounting for the total fatty acid is shown in Table 3.

TABLE 2
Fatty acid compositions of Cinnamomum camphora seed kernel
oil, basa catfish basa catfish, and Perilla seed oil

| | Content (%, w/w) | | |
|---|---|---|---|
| Fatty | Camphor seed | Basa fish | Perilla |
| acid | kernel oil | oil stearin | seed oil |
| C8:0 | 0.62 ± 0.05 | 0.23 ± ′0.10 | ND |
| C10:0 | 54.17 ± 0.09 | 0.01 ± 0.00 | ND |
| C11:0 | 0.15 ± 0.01 | ND | ND |
| C12:0 | 40.90 ± 0.12 | 0.13 ± 0.04 | ND |
| C13:0 | 0.26 ± 0.01 | 0.27 ± 0.01 | ND |
| C14:0 | 1.13 ± 0.01 | 5.67 ± 0.03 | 0.16 ± 0.03 |
| C15:0 | ND | 0.24 ± 0.00 | ND |
| C16:0 | 0.41 ± 0.01 | 45.76 ± 0.20 | 4.690.09 |
| C16:1 | ND | 1.18 ± 0.00 | 0.23 ± 0.02 |
| C17:0 | ND | 0.22 ± 0.00 | ND |
| C18:0 | 0.23 ± 0.01 | 10.09 ± 0.04 | 1.26 ± 0.06 |
| C18:1 | 1.81 ± 0.02 | 27.12 ± 0.11 | 21.85 ± 0.14 |
| C18:2 | 0.32 ± 0.01 | 6.69 ± 0.07 | 8.70 ± 0.10 |
| C18:3 | ND | 0.47 ± 0.04 | 62.93 ± 0.31 |
| C19:0 | ND | ND | ND |
| C19:1 | ND | ND | 0.14 ± 0.03 |
| C19:2 | ND | ND | ND |
| C20:0 | ND | 0.20 ± 0.01 | 0.04 ± 0.01 |
| C20:1 | ND | 0.75 ± 0.18 | ND |
| C20:2 | ND | 0.33 ± 0.00 | ND |
| C20:3 | ND | 0.25 ± 0.00 | ND |
| SFA | 97.87 ± 3.58 | 62.82 ± 3.66 | 6.15 ± 1.31 |
| USFA | 2.13 ± 0.24 | 37.18 ± 2.33 | 93.85 ± 3.52 |

TABLE 3
Base oil for functional food oils and fats with different
percentage of medium-chain fatty acids in total fatty acids
Fatty acid compositions of base oil (L/Ln = 0.5)

| Fatty | Content (%, w/w) | | | |
|---|---|---|---|---|
| acid | BO1 | BO2 | BO3 | BO4 |
| C8:0 | 0.47 ± 0.01 | 0.48 ± 0.02 | 0.49 ± 0.02 | 0.49 ± 0.01 |
| C10:0 | 35.55 ± 1.52 | 36.68 ± 2.13 | 37.81 ± 1.85 | 38.94 ± 2.33 |
| C11:0 | 0.10 ± 0.02 | 0.10 ± 0.04 | 0.10 ± 0.01 | 0.11 ± 0.02 |
| C12:0 | 26.87 ± 1.43 | 27.72 ± 2.11 | 28.57 ± 1.63 | 29.42 ± 1.77 |
| C13:0 | 0.24 ± 0.05 | 0.24 ± 0.03 | 0.24 ± 0.03 | 0.24 ± 0.04 |
| C14:0 | 2.24 ± 0.37 | 2.17 ± 0.25 | 2.11 ± 0.31 | 2.04 ± 0.24 |
| C15:0 | 0.06 ± 0.01 | 0.06 ± 0.01 | 0.06 ± 0.02 | 0.05 ± 0.01 |
| C16:0 | 12.64 ± 1.23 | 11.90 ± 0.88 | 11.16 ± 1.36 | 10.41 ± 1.03 |
| C16:1 | 0.33 ± 0.11 | 0.31 ± 0.08 | 0.29 ± 0.06 | 0.27 ± 0.09 |
| C17:0 | 0.06 ± 0.01 | 0.05 ± 0.02 | 0.05 ± 0.01 | 0.05 ± 0.01 |
| C18:0 | 2.90 ± 0.3 3 | 2.74 ± 0.45 | 2.57 ± 0.38 | 2.41 ± 0.26 |
| C18:1 | 10.08 ± 0.55 | 9.58 ± 0.73 | 9.08 ± 1.02 | 8.58 ± 0.94 |
| C18:2 | 2.67 ± 0.32 | 2.53 ± 0.65 | 2.39 ± 0.25 | 2.25 ± 0.34 |
| C18:3 | 5.27 ± 0.45 | 4.95 ± 1.13 | 4.63 ± 0.58 | 4.31 ± 0.69 |
| C19:1 | 0.01 ± 0.00 | 0.01 ± 0.00 | 0.01 ± 0.00 | 0.01 ± 0.00 |
| C20:0 | 0.06 ± 0,01 | 0.05 ± 0.01 | 0.05 ± 0.02 | 0.05 ± 0.01 |
| C20:1 | 0.20 ± 0.05 | 0.18 ± 0.06 | 0.17 ± 0.03 | 0.16 ± 0.02 |
| C20:2 | 0.09 ± 0.02 | 0.08 ± 0.01 | 0.08 ± 0.03 | 0.07 ± 0.01 |
| C20:3 | 0.07 ± 0.01 | 0.06 ± 0.01 | 0.06 ± 0.01 | 0.05 ± 0.01 |
| SFA | 81.18 ± 3.49 | 82.19 ± 2.11 | 83.21 ± 2.33 | 84.22 ± 3.12 |
| USFA | 18.82 ± 2.45 | 17.81 ± 1.99 | 16.79 ± 2.15 | 15.78 ± 2.37 |

Note:
L/Ln refers to the mass ratio of linoleic acid to linolenic acid.

C57BL/6 male mice aged 3-4 weeks with a body weight of 13-16 g were selected for the experiment. During the experiment, the mice were fed in a standard breeding cage with free access to food and water, 12h/12h day and night cycle light, the rearing temperature was 23±2° C., and the humidity was 40%-60%. After a week of adaptive feeding, the mice were randomly divided into two groups, 10 mice were divided in the normal chow group (Normal Chow, NC group) and fed with normal chow AIN-93M, and 60 mice were divided in the high-fat feed group (HFD group) and fed with high-fat feed D12451. The mice were weighed and the body weight of mice was recorded after feeding for 8 weeks. Mice in the HFD group whose body weight was 20% or more than the average body weight of the mice in the NC group were selected as the nutritionally obese model mice and used for subsequent experiments.

After the modeling was completed, the nutritionally obese model mice were randomly divided into 6 groups based on the body weight, namely the HFD group, recovery group (NR group) and 4 base oil for functional food oils and fats groups (BO1 group, BO2 group, BO3 group, and BO4 group), and the mice were continued feeding for 10 weeks. The mice in the HFD group continued to be fed with high-fat feed, the NR group were fed with normal chow, and the mice in BO1, BO2, BO3, and BO4 groups were fed with high-fat feed containing base oil for functional food oils and fats (H-BO) with medium-chain fatty acids at a mass percentage being 63%, 65%, 67%, and 69%, respectively, and the mass ratio of linoleic acid to linolenic acid in long-chain fatty acids being 0.5. The mice in the NC group continued to be fed with normal chow AIN-93M until the end of the experiment. See Table 1-1 and Table 1-2 for specific feed formula of the feed used in the experiment.

At the end of the experiment, the mice was weighed and the final body weight was recorded, the eyeballs were removed and the blood was collected, and the serum was separated to measure the indicators such as triglyceride (TG), total cholesterol (TC), low-density lipoprotein cholesterol (LDL-C), and high-density lipoprotein cholesterol (HDL-C) fasting blood glucose (FBG), insulin (FINs), alanine aminotransferase (also known as ALT), and aspartate aminotransferase (also known as AST) in the mouse serum. The peritesticular fat and the perirenal fat of the mice were separated and weighed, and the sum of the weight of the peritesticular fat and the weight of the perirenal fat was used as the mass of the abdominal fat. The fat coefficient (the percentage of fat accounting for the body weight) and homeostatic model insulin resistance index (HOMA-IR=[(FBG(mmol/L)×FINs(ng/ml)]/22.5) were calculated.

Statistical software package SPSS 19.0 (SPSS Inc., Chicago, IL, USA) was used for data processing. The results for the animal experiment are shown in FIG. 1A-FIG. 1D to FIG. 3A-FIG. 3C.

From FIG. 1A to FIG. 1B, it can be seen that the fat coefficients of mice in normal chow group (NC group), recovery group (NR group), BO1 group, BO2 group, BO3 group, and BO4 group was at a low level, and the fat coefficient of mice in BO1 group, BO2 group, BO3 group and BO4 group was 20.6%, 24.3%, 24.8% and 25.3% lower than that of the mice in the high-fat feed group (HFD group); respectively, indicating that the base oil for functional food oils and fats, in which the medium-chain fatty acids accounted for 63%, 65%, 67%, and 69%, and the mass ratio of linoleic acid in long-chain fat to linolenic acid in long-chain fat was 0.5, had an effect in significantly reducing the body fat of the mice.

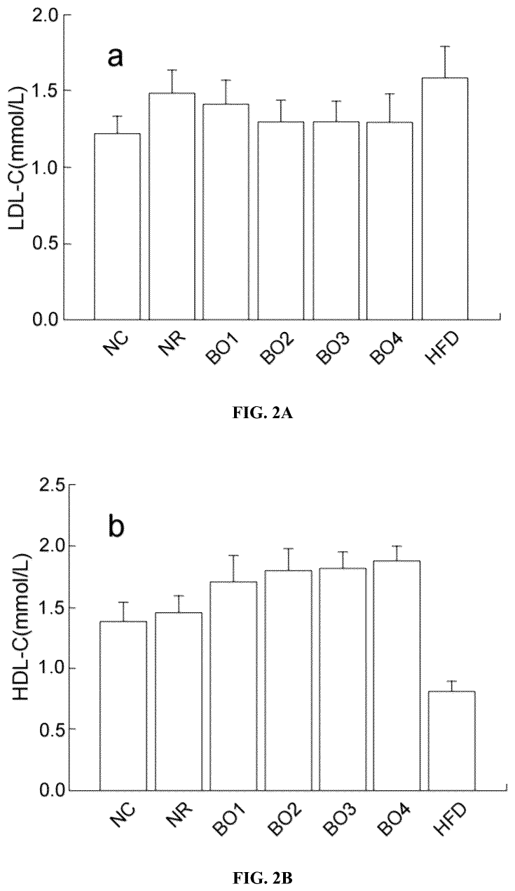

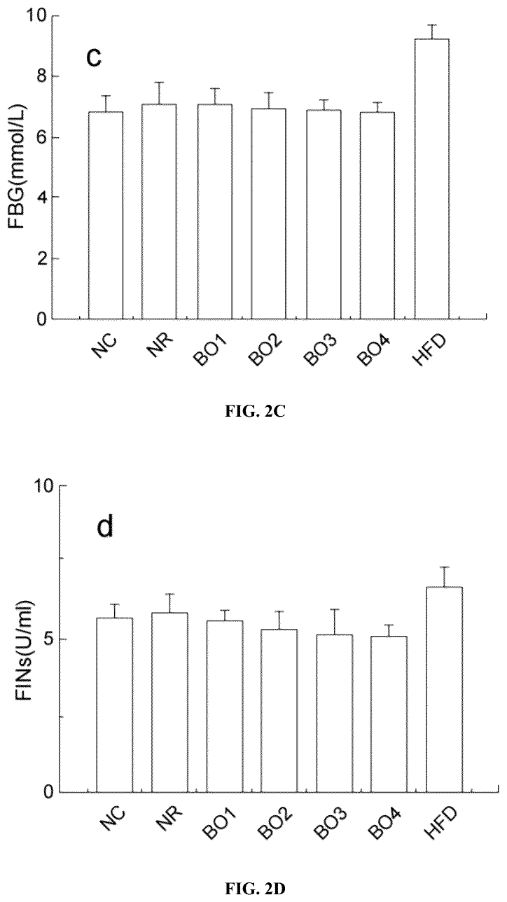

From FIG. 1C to FIG. 2B, it can be seen that base oil for functional food oils and fats had a great effect on serum TG, serum TC and serum LDL-C levels of the mice. The TG, TC and LDL-C levels of mice in the normal chow group (NC group), the recovery group (NR group), the BO1 group, the BO2 group, BO3 group, and the BO4 group were all at a low level, and the TG, TC and LDL-C levels of mice in the BO1 group were 16.8%, 12.1%, and 11.3% lower than that of the mice in the high-fat feed group (HFD group), and the levels of TG, TC and LDL-C in the mice in the BO2 group were 22.7%, 14.0%, and 18.0% lower than those in the HFD group, the TG, TC and LDL-C levels of the mice in the BO3 group were 24.3%, 18.9%, 18.0% lower than those in the high-fat feed group (HFD group), and the TG, TC and LDL-C levels of the mice in the BO4 group were 25.6%, 19.4%, and 18.6% lower than those in the high-fat feed group (HFD group). This indicated that the base oil with the medium-chain fatty acids accounting for 65%, 67%, and 69% by mass, and the mass ratio of linoleic acid in the long-chain fatty acids to linolenic acid in the long-chain fatty acids being 0.5 had an effect of significantly reducing blood lipids in mice.

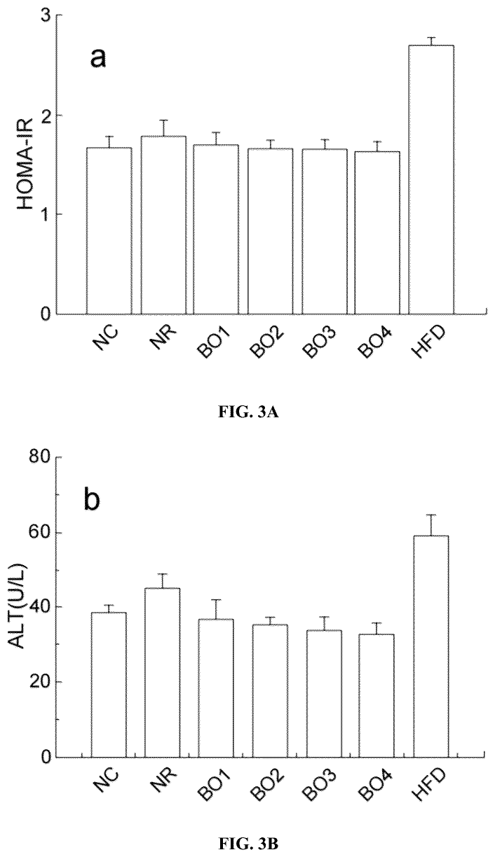

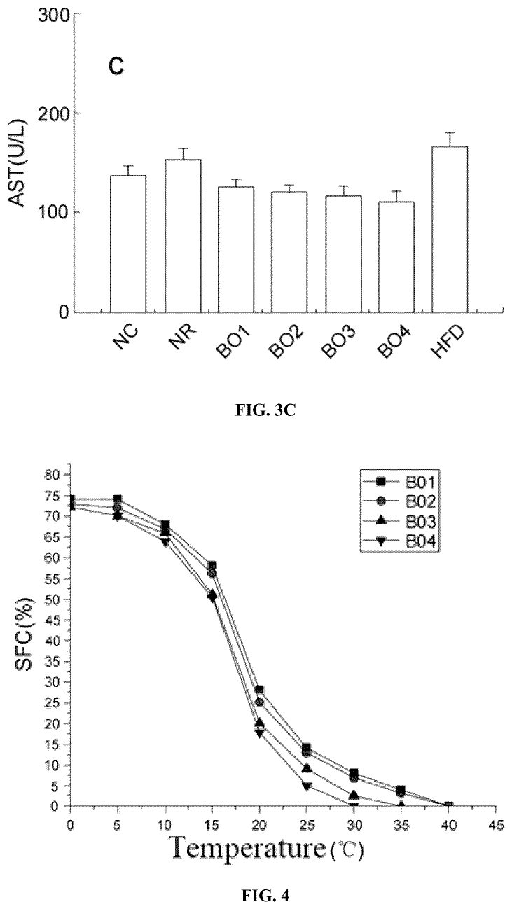

Glucose metabolism is closely correlated to fat metabolism, and disorder of fat metabolism can easily lead to disorder of glucose metabolism. From FIG. 2C to FIG. 3A, it can be seen that the FBG level, the FINs level and the HOMA-IR index in the serum of mice in the normal chow group (NC), the recovery group (NR) and the BO4 group, the BO3 group, the BO2 group, and the BO1 group were at a normally low level, no significant difference was present, and the FBG level, the FINs level and the HOMA-IR index of BO4 group mice were 26.0%, 24.2%, 39.2% lower than those of high-fat feed group (HFD) mice, the FBG level, the FINs level and the HOMA-IR index of mice in the BO3 group were 25.4%, 23.3%, and 38.6% lower than those of high-fat feed group (HFD) mice, and the FBG level, the FINs level and the HOMA-IR index of BO2 group mice were 25.2%, 21.2%, 38.3% lower than those of the mice in the high-fat feed group (HFD), the FBG level, the FINs level and the HOMA-IR index of mice in the BO1 group were 23.8%, 16.7%, and 36.9% lower than the mice in the high-fat feed group (HFD), respectively; This indicated that base oil with a mass percentage of medium-chain fatty acid being 63%, 65%, 67%, and 69% and a mass ratio of linoleic acid to linolenic acid in long-chain fatty acids being 0.5 can significantly improve glucose metabolism in mice.

It can be seen from FIG. 3B and FIG. 3C that base oil for functional food oils and fats has a great effect on the serum ALT and AST levels of mice. The ALT and AST levels of mice in the normal chow group (NC group), the recovery group (NR group), the BO4 group, the BO3 group, the BO2 group and the BO1 group were all at lower levels, and the ALT and the AST levels of mice in the BO4 group were 45.3% and 33.5% lower than the mice in the high-fat feed group (HFD group) mice, respectively; and the ALT and the AST levels of the mice in the BO3 group were 43.1% and 30.0% lower than the mice in the high-fat feed group (HFD group) mice respectively; the ALT and the AST levels of the mice in the BO2 group were 40.7%, 27.9% lower than the mice in the high-fat feed group (HFD group), and the ALT and the AST levels of the mice in the BO1 group were 37.9% and 24.5% lower than the mice in the high-fat feed group (HFD group), indicating that the base oil for functional food oils and fats with the mass percentage of the medium-chain fatty acids and long-chain fatty acids being 63%, 65%, 67%, and 69% and a mass ratio of linoleic acid to linolenic acid in the long-chain fatty acids being 0.5 can significantly repair liver damage in mice.

From the comprehensive analysis of FIG. 1A-FIG. 1D to FIG. 3A-FIG. 3C, it can be concluded that the base oil for functional food oils and fats with the mass percentage of medium-chain fatty acids being between 63% and 69%, and the mass ratio of linoleic acid to linolenic acid in long-chain fatty acids being 0.5 has the effect of improving glucose and lipid metabolism disorder in mice. Among them, when the mass percentage of medium-chain fatty acids was 65% to 69% and the mass ratio of linoleic acid to linolenic acid in long-chain fatty acids was 0.5, the base oil for functional food oils and fats has the most significant effect on improving glucose and lipid metabolism disorder in mice.

From FIG. 1A-FIG. 1D to FIG. 4, the melting range of the base oil for functional food oils and fats and the effect of improving the fat metabolism disorder in mice and efficiently supplementing the essential fatty acids and functional fatty acids in the body were comprehensively evaluated. When the medium-chain fatty acid in the base oil for functional food oils and fats accounted for 65% to 69% of the total mass of the fatty acids, the base oil for functional food oils and fats had a significant effect on improving the disorder of fat metabolism in mice and efficiently supplementing essential fatty acids and functional fatty acids in the body. Among them, base oil for functional food oils and fats in which the medium-chain fatty acids accounted for 65% by mass of the total fatty acids and the SFC was 13.8% at 25° C. and 7.5% at 30° C. had a wide melting range. Therefore, it was most preferable to choose base oil for functional food oils and fats in which the mass percentage of medium-chain fatty acids accounts for the total fatty acid is 65%.

### Example 2

In this Example, according to the mass percentage of medium-chain fatty acids to the total fatty acid being 65%, and the mass ratio of linoleic acid to linolenic acid being 0.5, a mixed oil consisting of 164.06 g of Cinnamomum camphora seed kernel oil, 65.48 g of basa catfish solid fraction and 20.46 g of Perilla seed was placed in 4 reactors of the same specification, and immobilized lipase Novozyme 435, immobilized lipase Staphylococcus caprae lipase, immobilized lipase Lipozyme RM IM, and immobilized lipase Lipozyme TL IM were respectively added in 4 reactors in an amount of 10% (w/w) of the mass of the mixed oil. The reaction conditions for ternary transesterification were as follows: magnetic stirring (stirrer bar 30 mm×10 mm, rotation speed 100 rpm) was used, the optimum temperature recommended for each lipase was selected as the reaction temperature (which was individually 60° C. for immobilized lipase Novozyme 435, immobilized lipase Lipozyme RM IM, immobilized lipase Lipozyme TL IM; and 50° C. for immobilized lipase Staphylococcus caprae lipase), and the reaction time was 4 hours.

After the ternary transesterification reaction was completed, the ternary transesterification rate was measured by a detection method of HPLC-ELSD. The effect of lipase species on ternary transesterification rate was compare and analyzed for purpose of selection of lipase species. It can be seen from Table 4 that when lipase Staphylococcus caprae lipase was used to prepare base oil for functional food oils and fats, the ternary transesterification rate was the highest, reaching 74.34% (w/w), and the lipase providing the highest catalytic efficiency was Staphylococcus caprae lipase.

TABLE 4
Effect of lipase species on ternary transesterification rate

| Catalyst | Lipozyme | Lipozyme | Novozyme | Staphylococcus |
|---|---|---|---|---|
| species | RM IM | TL IM | 435 | caprae lipase |
| Ternary | 69.88 | 71.34 | 72.67 | 74.34 |
| transesterification | | | | |
| rate (w/w %) | | | | |

### Example 3

In this Example, based on the mass percentage of medium-chain fatty acids accounting for the total fatty acid being 65%, and the mass ratio of linoleic acid to linolenic acid being 0.5, 164.06 g of Cinnamomum camphora seed kernel oil, 65.48 g of basa catfish solid fraction and 20.46 g of Perilla seed oil were weighed and placed in a reactor. The reaction conditions for ternary transesterification were as follows: lipase Staphylococcus caprae lipase was 5% to 25% (percentage based on the mass of the mixed oil), magnetic stirring (stirring bar 30 mm×10 mm, speed 100 rpm) was used, reaction temperature was 50° C., and the reaction time was 4 hours.

Upon the completion of the reaction, the ternary transesterification rate was determined by a detection method of HPLC-ELSD. The effect of enzyme addition amount on the ternary transesterification rate was compare and analyzed, and the enzyme addition amount was determined. It can be seen from table 5 that when the enzyme addition amount was 10%, the ternary transesterification rate was the highest, reaching 74.21% (w/w), and the optimum enzyme addition amount was 10%.

TABLE 5
Effect of addition amount of Staphylococcus caprae
lipase on ternary transesterification rate

| | Enzyme quantity | | | | |
|---|---|---|---|---|---|
| | (w/w %) | | | | |
| | 5 | 10 | 15 | 20 | 25 |
| ternary | 69.78 | 74.21 | 72.49 | 71.13 | 60.65 |
| transesterification rate | | | | | |
| (w/w %) | | | | | |

### Example 4

In this Example, based on the mass percentage of medium-chain fatty acids accounting for the total fatty acid being 65% and the mass ratio of linoleic acid to linolenic acid being 0.5, 164.06 g of Cinnamomum camphora seed kernel oil, 65.48 g of basa catfish solid fraction and 20.46 g of Perilla seed oil were weighed and placed in a reactor. The reaction conditions for ternary transesterification were as follows: lipase Staphylococcus caprae lipase 10% (mass of the mixed oil percentage) was added, magnetic stirring (stirrer 30 mm×10 mm, speed 100 rpm) was performed, the reaction temperature was 35-55° C., and the reaction time was 4 hours.

Upon the completion of the reaction, the ternary transesterification rate was determined by a detection method of HPLC-ELSD. The effect of reaction temperature on ternary transesterification rate was compared and analyzed, and the reaction temperature was determined. It can be seen from Table 6 that when the reaction temperature was 50° C., the ternary transesterification rate was the highest, reaching 74.33% (w/w), and the optimum reaction temperature was 50° C.

TABLE 6
Effect of transesterification temperature
on ternary transesterification rate

| | Temperature (° C.) | | | | |
|---|---|---|---|---|---|
| | 35 | 40 | 45 | 50 | 55 |
| ternary | 66.68 | 68.76 | 71.88 | 74.33 | 72.54 |
| transesterification rate | | | | | |
| (w/w %) | | | | | |

### Example 5

In this Example, based on the mass percentage of medium-chain fatty acids accounting for the total fatty acid being 65%, and the mass ratio of linoleic acid to linolenic acid being 0.5, 164.06 g of Cinnamomum camphora seed kernel oil, 65.48 g of basa catfish solid fraction and 20.46 g of Perilla seed oil were weighed and placed in a reactor. The reaction conditions ternary transesterification were as follows: lipase Staphylococcus caprae lipase 10% (percentage based on the mass of the mixed oil) was added, magnetic stirring (stirrer bar 30 mm×10 mm, speed 100 rpm) was performed, the reaction temperature was 50° C., and the reaction time was 1-8 hours.

Upon the completion of the reaction, the ternary transesterification rate was determined by a detection method of HPLC-ELSD. The effect of reaction time on ternary transesterification rate was compared and analyzed, and the reaction time was determined. It can be seen from Table 7 that when the reaction time was 4h, the ternary transesterification rate was the highest reaching 74.36% (w/w), and the optimum reaction time was 4 hours.

TABLE 7
Effect of transesterification time on ternary transesterification rate

| | Ternary |
|---|---|
| | transesterification |
| Reaction time (h) | rate (w/w %) |
| 1 | 35.82 |
| 2 | 64.92 |
| 3 | 71.83 |
| 4 | 74.36 |
| 5 | 72.46 |
| 6 | 72.39 |
| 7 | 71.44 |
| 8 | 71.12 |

### Example 6

In this Example, based on the mass percentage of medium-chain fatty acids accounting for the total fatty acid being 65%, and the mass ratio of linoleic acid to linolenic acid being 0.5, 164.06 g of Cinnamomum camphora seed kernel oil, 63.62 g of palm stearin and 22.32 g of linseed oil were weighed and placed in a reactor. The reaction conditions for ternary transesterification were as follows: lipase Staphylococcus caprae lipase 10% (percentage based on the mass of the mixed oil) was added, magnetic stirring (stirrer bar 30 mm×10 mm, speed 100 rpm) was performed, the reaction temperature was 50° C., and the reaction time was 4 hours.

Upon the completion of the reaction, the ternary transesterification rate was determined to be 74.34% by a detection method of HPLC-ELSD, and the content of fatty acids in base oil for functional food oils and fats was determined by GC. The results were: caprylic acid 0.49%, capric acid 36.71%, lauric acid 27.56%, linoleic acid 2.52%, and linolenic acid 4.98%.

### Example 7

Functional non-dairy creamer was prepared using the base oil for functional food oils and fats prepared in respective examples as well as other ingredients, and the specific operation steps of the preparation process were as follows:
(1) Preparing material liquid, comprising: weighing a mass of water-soluble substances according to the functional non-dairy creamer formula Table 8, and placing the a water-soluble substance in hot water at 63-67° C., and weighing a mass of base oil for functional food oils and fats and mono- and di-glyceride, dissolving the base oil for functional food oils and fats and mono- and di-glyceride in aqueous solution after all the water-soluble substances were dissolved, stirring the resulting mixture at 60-90 rpm for 25-30 min;
(2) Emulsification by shearing, comprising: shearing a material liquid for about 1 to 2 minutes using a shearing machine;
(3) Homogeneous emulsification, comprising: homogenizing the material liquid twice under a pressure of 25-30 Mpa using a sterilized homogenizer;
(4) Drying and granulation, comprising: performing drying and granulation using a pressure sprayer and a fluidized bed, with an inlet air temperature of 180° C. and an outlet air temperature of 90-100° C.

TABLE 8
Formula of functional non-dairy creamer

| Ingredients | Mass percentage (%) |
|---|---|
| Base oil for functional food oils and fats | 20.0-50.0 |
| Starch syrup | 40.0-70.0 |
| Skimmed milk powder | 5.0-10.0 |
| Mono- and di- glyceride | 0.5-5.0 |
| Sodium tripolyphosphate | 0.1-5.0 |
| Sodium caseinate | 0.1-5.0 |
| Hydroxymethyl cellulose | 0.2-0.6 |
| Hexametaphosphate | 0.1-1.5 |
| Dipotassium phosphate | 0.1-5.0 |
| Sodium citrate | 0.1-0.5 |
| Sodium chloride | 0.0-0.5 |
| Food flavor | 0.0-0.5 |
| SiO₂ | 0.0-0.5 |
| Total | 100.0 |

### Example 8

Functional margarine was prepared by using the products prepared with the base oil for functional food oils and fats in each Examples and other ingredients, and the specific operation steps of the preparation process were as follows:
(1) Preparation of oil phase, comprising: weighing a mass of base oil for functional food oils and fats, lecithin, and tripolyglycerol esters of fatty acids according to the functional margarine formula in Table 9 and heating the resulting mixture to 65° C., stirring and dissolving the mixture to obtain an oil phase;
(2) Preparation of water phase, comprising: weighing a quality of purified water, casein and sweetener, and stirring the resulting mixture evenly at 65° C. to prepare a water phase;
(3) Emulsification by shearing, comprising: mixing the oil phase and the water phase evenly at 65° C., and shearing the resulting material liquid for about 1 to 2 minutes using a shearing machine;
(4) quenching and molding by kneading, comprising: stirring the emulsion in an ice bath at 350 rpm for 5 min;
(5) Aging, comprising: transferring the emulsion to a 20° C. incubator for 24 hours of aging, then refrigerating and aging the emulsion at 4° C. for 24 hours to obtain functional margarine.

TABLE 9
Functional margarine formula

| Ingredients | Mass percentage (%) |
|---|---|
| Base oil for functional food oils and fats | 50.0-60.0 |
| Lecithin | 2.5-5 |
| Tripolyglycerol esters of fatty acids | 2.5-5 |
| Casein | 0.01-0.1 |
| Sweetener | 0.1-1 |
| Purified water | 30-35 |
| Total | 100.0 |

1. A base oil for functional food oils and fats, wherein base oil for functional food oils and fats is formed through ternary transesterification of a medium-chain triglyceride, a high-melting-point fat, and an oil rich in linolenic acid; wherein the medium-chain triglyceride is a Cinnamomum camphora seed kernel oil, or mixed oils and fats having similar components to fatty acids in the Cinnamomum camphora seed kernel oil; the high-melting-point fats are fats with a melting point ranging from 44° C. to 52° C.; and the oil rich in linolenic acid is Perilla seed oil or linseed oil;
wherein the medium-chain fatty acids accounts for 63% to 69% by mass based on mass of the fatty acid, and a mass ratio of linoleic acid to linolenic acid in the mass of long-chain fatty acid is 0.5; wherein the medium-chain fatty acid is derived from the Cinnamomum camphora seed kernel oil or mixed oils and fats having similar components to the fatty acids in the Cinnamomum camphora seed kernel oil; and the long-chain fatty acid is derived from a fat having a melting point of 44° C.-52° C. and an oil rich in linolenic acid.

2. (canceled)

3. (canceled)

4. The base oil for functional food oils and fats according to claim 1, wherein the fat having a melting point of 44° C.-52° C. is basa catfish solid fraction or palm stearin.

5. The base oil for functional food oils and fats according to claim 1, wherein the medium-chain fatty acid in the base oil for functional food oils and fats accounts for 65% by mass of the total fatty acid.

6. A method for preparing the base oil for functional food oils and fats of claim 1, comprising the following steps: subjecting medium-chain triglycerides and high-melting point fats, oils rich in linolenic acid to ternary transesterification at a proper temperature and at a proper strength of stirring, using lipase as a catalyst, to obtain the base oil for functional food oils and fats at one step; the medium-chain fatty acids accounts for 65% by mass, and a mass ratio of linoleic acid to linolenic acid is 0.5; the lipase is selected from the group consisting of Lipozyme RM IM, Lipozyme TL IM, Novozyme 435, and Staphylococcus caprae lipase; the addition amount of lipase is 5-25% of the mass of the mixed oil, a reaction temperature for ternary transesterification is 35-55° C., and a reaction time for ternary transesterification is 1-8h.

7. The method according to claim 6, wherein the addition amount of lipase is 10% by mass based on the mass of the mixed oil, the reaction temperature for ternary transesterification is 50° C., and the reaction time for ternary transesterification is 4h.

8. (canceled)

9. (canceled)

10. (canceled)

11. (canceled)

12. (canceled)
